(19) United States
(12) Patent Application Publication
(10) Pub. No.: US 2010/0040695 A1
(43) Pub. Date: Feb. 18, 2010

(54) METHODS FOR PROVIDING NUTRITIONAL PRODUCTS FOR CUSTOMIZED ENERGY INTAKE

(75) Inventors: Marie Georgina Feuvrier-Roy, Chaux-Neuve (FR); Donald Joseph Leonard, Los Angeles, CA (US); Ankur Mittal, Lutry (CH)

Correspondence Address: K&L Gates LLP, P.O. Box 1135, CHICAGO, IL, 60690, US

(73) Assignee: NESTEC S.A., Vevey (CH)

(21) Appl. No.: 12/540,561

(22) Filed: Aug. 13, 2009

Related U.S. Application Data
(60) Provisional application No. 61/088,553, filed on Aug. 13, 2008.

(51) Int. Cl. A61K 9/14; A61K 38/16; A61P 39/00

(52) U.S. Cl. 424/490; 514/12; 424/489

(57) ABSTRACT

Methods utilizing bite-size nutritional products for customized protein intake are provided. In a general embodiment, the present disclosure provides a method of providing nutrition to an athlete. The method comprises providing a bite-size protein-based product and providing personalized guidelines for consuming the bite-size protein-based product. The personalized guidelines provide the recommended amount of the bite-size protein-based product to consume before exercising, during exercising and/or during recovery after exercising based on one or more characteristics of the athlete and/or one or more training regimens of the athlete.

### CROSS REFERENCE TO RELATED APPLICATIONS

The following commonly-owned co-pending patent application entitled “BITE-SIZE CARBOHYDRATE NUTRITIONAL PRODUCTS AND METHODS FOR USING SAME”, U.S. Provisional Patent Application Ser. No. 61/088,559 filed on Aug. 13, 2008, concurrently filed herewith is hereby incorporated herein by reference in its entirety.

### PRIORITY CLAIM

This application claims the benefit of U.S. Provisional Patent Application Ser. No. 61/088,553 filed on Aug. 13, 2008, the entire disclosure of which is hereby incorporated by reference.

### BACKGROUND

The present disclosure generally relates to methods for providing nutrition before, during and/or after exercising. More specifically, the present disclosure is directed to methods for providing bite-size nutritional products for customized protein intake before, during and/or after exercising.

Nutritional products for providing protein to a consumer are known. Typically, these nutritional products are in the shape of large bars and are bulky to consume. Moreover, these nutritional products are not associated with any recommended intake requirements based on the types of consumers or activities of the consumers. For example, an athlete who engages in endurance activities will likely need to eat a greater amount of the nutritional product than a person going for a casual walk. As a result, a person who eats the nutritional products does not know how much of the nutritional product to consume in order to optimize the protein providing contents of the nutritional product.

### SUMMARY

The present disclosure provides various methods for providing bite-size nutritional products for customized protein intake before, during and/or after exercising. In a general embodiment, the present disclosure provides a method of providing nutrition to an athlete. The method comprises providing a bite-size protein-based product and providing personalized guidelines for consuming the bite-size protein-based product. The personalized guidelines provide the recommended amount of the bite-size protein-based product to consume before exercising, during exercising and/or during recovery after exercising based on one or more characteristics of the athlete and/or one or more training regimens of the athlete.

In an embodiment, the characteristic(s) of the athlete is weight, height, age, gender or a combination thereof.

In an embodiment, the training regimen(s) is aerobics, weight-lifting, running, swimming, cycling or any other suitable individual or group sports.

In an embodiment, the personal guidelines are further based on the duration of the training regiment of the athlete.

In an embodiment, the training regimen is determined based on a sport.

In an embodiment, the training regimen is determined based on a personal objective.

In an embodiment, the personal objective is weight-loss, muscle building or a combination thereof.

In an embodiment, the personalized guidelines provide the recommended amount of the bite-size protein-based product to consume during exercising based on at least one of the characteristic of the athlete and the training regimen of the athlete.

In an embodiment, the bite-size protein-based product has a substantially spherical shape.

In an embodiment, the bite-size nutritional product has a sweet taste and is highly concentrated in protein.

In an embodiment, the bite-size protein-based product comprises a coating.

In an embodiment, the coating comprises chocolate.

In an embodiment, the coating comprises particulates.

In an embodiment, the bite-size protein-based product is in the shape of a sphere having a diameter ranging from about 12 mm to about 35 mm.

In an embodiment, the bite-size protein-based product weighs from about 5 grams to about 25 grams.

In an embodiment, the bite-size protein-based product comprises a protein content of about 2 grams to about 4 grams.

In an embodiment, the bite-size protein-based product comprises a protein blend comprising soy protein isolates, whey protein isolates and calcium caseinate.

In an embodiment, the bite-size protein-based product comprises an amount of protein ranging from about 20% to about 40% by weight of the bite-size protein-based product.

In an embodiment, the bite-size protein-based product comprises an amount of fat ranging from about 10% to about 40% by weight of the bite-size protein-based product.

In an embodiment, the bite-size protein-based product comprises an amount of protein above 15% by weight together with less than 10% saturated fat by weight of the protein-based product.

In another embodiment, the present disclosure provides a method of selling a bite-size protein-based product. The method comprises providing a plurality of bite-size protein-based products in a package and providing personalized guidelines for consuming the bite-size protein-based products. The personalized guidelines provide the recommended amount of the bite-size protein-based product to consume before exercising and during recovery after exercising. The method further comprises selling the bite-size protein-based product package and the guidelines to an athlete.

In an embodiment, the personalized guidelines are based on at least one of a characteristic of the athlete and a training regimen of the athlete.

In an embodiment, the bite-size protein-based products are not individually wrapped.

In an embodiment, the bite-size protein-based products are individually wrapped.

In an alternative embodiment, the present disclosure provides a nutritional kit comprising a plurality of bite-size protein-based product and personalized guidelines on how many of the bite-size protein-based products an athlete should consume before exercising and during recovery after exercising.

In an embodiment, a plurality of the bite-size protein-based products and the personalized guidelines are together in a package.

In an embodiment, the personalized guidelines provide the recommended amount of the bite-size protein-based product to consume before exercising and during recovery after exercising based on the body weight of the athlete.

In an embodiment, the personalized guidelines provide the recommended amount of the bite-size protein-based product to the athlete before exercising and during recovery after exercising based on the training regimen of the athlete.

In another embodiment, the present disclosure provides a method of enhancing the performance of an athlete. The method comprises providing a bite-size protein-based product and providing personalized guidelines for consuming the bite-size protein-based product. The personalized guidelines provide the recommended amount of the bite-size protein-based product to consume before the performance based on at least one of a characteristic of the athlete and a training regimen of the athlete.

In an alterative embodiment, the present disclosure provides a method of improving recovery of an athlete after exercising. The method comprises providing a bite-size protein-based product and providing personalized guidelines for consuming the bite-size protein-based product. The personalized guidelines provide the recommended amount of the bite-size protein-based product to consume during recovery after exercising based on at least one of a characteristic of the athlete and a training regimen of the athlete.

An advantage of the present disclosure to provide improved methods of providing nutritional products before, during and/or after exercising.

Another advantage of the present disclosure is to provide new methods for customized protein intake before, during and/or after exercising.

Still another advantage of the present disclosure is to provide an improved method for providing protein to an athlete before, during and/or after exercising.

Additional features and advantages are described herein, and will be apparent from, the following Detailed Description.

### DETAILED DESCRIPTION

The present disclosure relates generally to methods for providing nutrition to an athlete before, during and/or after exercising. For example, bite-size nutritional products can be specifically designed and provided to an athlete for customized protein intake and portion control. The bite-size nutritional products can have a sweet taste and be highly concentrated in protein. In addition, the bite-size nutritional products can meet both qualitative (e.g. a tri-source protein blend) and quantitative (e.g. fixed and regular amounts of protein per bite-size) protein requirements.

In a general embodiment, the present disclosure provides a method of providing nutrition to an athlete using customized or personal guidelines in conjunction with bite-size protein-based products. The method comprises providing a bite-size protein-based product and providing personalized guidelines for consuming the bite-size protein-based product. The personalized guidelines provide the recommended amount (e.g. how many) of the bite-size protein-based product to consume before exercising, during exercising and/or during recovery after exercising based on one or more characteristics of the athlete and/or one or more training regimens of the athlete. The personal guidelines can also be based on the duration of the training regiment of the athlete.

The bite-size protein-based products allow for personalized and optimal dosing of protein for providing protein to the athlete because of the size of the nutritional products. For example, the athlete can consume a specified number of bite-size protein-based products to provide the right amount of protein to the athlete as set forth in the personalized guidelines. In this manner, the portions of the bite-size protein-based product consumed by the athlete can be easily determined and monitored. In an embodiment, the intake amount (e.g. amount consumed) is guided by the characteristic(s) of the athlete, and the personalized guidelines specify two dosage amounts of the bite-size protein-based products for the athlete to consume—one dosage amount before exercising and another dosage amount after exercising during the recovery period.

To prepare the guidelines and determine what guidelines should be followed, the one or more characteristics of the athlete can be, by way of example, weight, height, age, gender or a combination thereof. Any other physical characteristics of the athlete can also be used for determining the guidelines. The one or more training regimens can include, by way of example, aerobics, weight-lifting, running swimming, cycling or any other suitable individual or group sports. The training regimen(s) can also be determined based on any type of sport (e.g. baseball, football, basketball, soccer, etc.).

The training regimen(s) can be determined based a personal objective of the athlete or consumer of the bite-size protein-based products. For example, the personal objective can be weight-loss, muscle building or a combination thereof.

In an embodiment, the personalized guidelines provide the recommended amount of the bite-size protein-based product for the athlete to consume during exercising based on at least one of the characteristic of the athlete and the training regimen of the athlete. The size of the bite-size protein-based product further provides ease of handling and dosing amount determination during the exercising.

In an embodiment, the bite-size protein-based product comprises a coating. The coating can be provided for convenient and non-sticky handling of the bite-size protein-based product during consumption. The coating can comprise chocolate, for example, in the form of a chocolate shell to prevent shape loss. In an alternative embodiment, the coating can comprise particulates.

The coating can comprise two different chocolates for visual differentiation. The coating can comprise different flavors such peanut butter, cookie dough and vanilla yoghurt. The coating can be added to a protein/fat core using any suitable methods such as, for example, panning or enrobing.

The bite-size protein-based product can have any suitable shape for ease of handling. In an embodiment, the bite-size protein-based product has a substantially spherical shape. For example, the bite-size protein-based product can be in the shape of a sphere having a diameter ranging from about 12 mm to about 35 mm. Preferably, the diameter of the sphere ranges from about 15 mm to about 25 mm.

In an embodiment, each of the bite-size protein-based products weighs from about 5 grams to about 25 grams. Preferably, each bite-size protein-based product can comprise a weight ranging from about 5 grams to about 15 grams. More preferably, each bite-size protein-based product can comprise a weight ranging from about 8 grams to 9 grams. A typical serving can include, for example, several of the bite-size protein-based products, which can provide a total of about 250 to about 320 kcal.

The distinct size of the bite-size protein-based products provides a portability to the bite-size protein-based products. In addition, as previously discussed, the size also makes it more convenient for the bite-size protein-based product to be eaten in discrete portion control amounts depending on the guidelines for the athlete. For example, the servings or the amount of the bite-size protein-based products that the athlete should consume before, during and/or after exercising can be easily adjusted depending the number of bite-size protein-based products needed for a specific protein requirement according to the guidelines.

The bite-size protein-based product can contain a discrete amount of protein in one or more bite-size products to provide any suitable amount of protein to an athlete. The percentage of energy (e.g. in the form of calories) coming from protein can range up to 30%.

In an embodiment, the bite-size protein-based product comprises an amount of protein ranging from about 20% to about 40% by weight of the bite-size protein-based product. In another embodiment, the bite-size protein-based product comprises an amount of protein of about 30% by weight of the bite-size protein-based product. Moreover, the bite-size protein-based product can be made so that there is a consistent and countable quantity of protein per single bite-size protein-based product, for example, between about 2 grams to about 4 grams per bite-size protein-based product. Preferably, the bite-size protein-based product comprises a protein content of about 2 grams to about 2.5 grams.

The bite-size protein-based product can also contain a discrete amount of fat in one or more bite-size products to provide any suitable amount of energy to an athlete. For example, each of the products can provide a fat amount up to 9 g/300 cal. Each of the products can also provide a saturated fat amount up to 4 g/300 cal. In an embodiment, the percentage of energy (e.g. in the form of calories) coming from fat can be up to about 25%.

In an embodiment, the bite-size protein-based product comprises an amount of fat ranging from about 10% to about 40% by weight of the bite-size protein-based product. Preferably, the bite-size protein-based product comprises an amount of fat of about 30% by weight of the bite-size protein-based product.

Specific ranges of the protein amounts in combination with the fat amounts of the bite-size protein-based product can also be advantageous in providing precise protein requirements to the athlete. In an embodiment, the bite-size protein-based product comprises an amount of protein above 15% by weight together with an amount of saturated fat that is less than 10% by weight of the protein-based product.

Any suitable proteins or blends of proteins can be used. For example, the bite-size protein-based product can comprise a protein blend comprising soy protein isolates, whey protein isolates and calcium caseinate. An example of the protein blend is the Tri-source™ protein blend.

The presence of proteins and/or fats in the nutritional product of the present disclosure has the advantage in that it is possible to provide an athlete with a more complete nutrition during performance. For the protein source, any suitable dietary protein may be such as, for example, animal proteins (e.g. milk proteins, meat proteins and egg proteins); vegetable proteins (e.g. soy protein, wheat protein, rice protein, and pea protein); mixtures of free amino acids; or combinations thereof. Milk proteins, such as casein and whey milk proteins, and soy proteins are particularly preferred.

The proteins may be intact or hydrolysed or a mixture of intact and hydrolysed proteins. It may be desirable to supply partially hydrolysed proteins (degree of hydrolysis between 2 and 20%), for example, for athletes believed to be at risk of developing cows' milk allergy. Generally, at least partially hydrolysed proteins are easier and faster to metabolize by the body. This is in particular true for amino acids. In an embodiment, the bite-size protein-based product contains single/essential amino acids such as, for example, L-leucine, L-valine and/or L-isoleucine.

The fat source has the advantage in providing for an improved mouth feel. Any fat source is suitable. For example, animal or plant fats may be used. To increase the nutritional value, n3-unsaturated and n6-unsaturated fatty acids may comprise the fat source. The fat source may also contain long chain fatty acids and/or medium chain fatty acids. For example, milk fat, canola oil, almond butter, peanut butter, corn oil and/or high-oleic acid sunflower oil may be used.

The bite-size protein-based products can be made using any suitable process (e.g. cold-extrusion). For example, the protein-based products can be made using a three step manufacturing process involving: 1) bite forming, 2) product handling after forming and 3) product enrobing (e.g. with chocolate or sugar coating).

The product forming step comprises making a roll of a protein/fat mass and cutting it into small pieces and forming them into spheres. Once formed, the product can be handled before enrobing (e.g. coating). Cooling down of the product is recommended to avoid product cold-flow. For example, special handling can be used to avoid the products from sticking together. The products can also be enrobed very quickly to avoid water picking and shape loss.

The applied coating can be a way to prevent stickiness, hold the shape of the center mass and provide a hard, shiny and non-melting ball. Different coating methods are possible such as a chocolate panning (e.g. for hard core) and hard sugar panning (e.g. for softer core).

In another embodiment, the present disclosure provides a method of selling a bite-size protein-based product. The method comprises providing a plurality of bite-size protein-based products in a package and providing personalized guidelines for consuming the bite-size protein-based products. The personalized guidelines provide the recommended amount of the bite-size protein-based product to consume before exercising and during recovery after exercising. The method further comprises selling the bite-size protein-based product package and the guidelines to an athlete. The personalized guidelines are based on one or more characteristics of the athlete and/or one or more training regimens of the athlete.

In an embodiment, the bite-size protein-based products are not individually wrapped. In another embodiment, the bite-size protein-based products are individually wrapped. Preferably, there are between 8 and 10 bite-sized protein-based products in each package in one embodiment.

In an alternative embodiment, the present disclosure provides a nutritional kit comprising a plurality of bite-size protein-based product and personalized guidelines on how many of the bite-size protein-based products an athlete should consume before exercising and during recovery after exercising. A plurality of the bite-size protein-based products and the personalized guidelines can be kept together in a package.

The personalized guidelines of the nutritional kit can provide the recommended amount of the bite-size protein-based product to consume before exercising and during recovery after exercising based on the body weight or other physical characteristics (e.g. weight, age, gender) of the athlete. In another embodiment, the personalized guidelines of the nutritional kit can provide the recommended amount of the bite-size protein-based product to the athlete before exercising and during recovery after exercising based on the training regimen of the athlete.

In another embodiment, the present disclosure provides a method of enhancing the performance of an athlete. The method comprises providing a bite-size protein-based product and providing personalized guidelines for consuming the bite-size protein-based product. The personalized guidelines provide the recommended amount of the bite-size protein-based product to consume before the performance based on at least one of a characteristic of the athlete and a training regimen of the athlete.

In an alterative embodiment, the present disclosure provides a method of improving recovery of an athlete after exercising. The method comprises providing a bite-size protein-based product and providing personalized guidelines for consuming the bite-size protein-based product. The personalized guidelines provide the recommended amount of the bite-size protein-based product to consume during recovery after exercising based on at least one of a characteristic of the athlete and a training regimen of the athlete.

### EXAMPLES

By way of example and not limitation, the following examples are illustrative of various embodiments of the present disclosure.

### Example 1

### Sample Formulations

TABLE 1
Bite-size protein-based product formulations #1 and #2

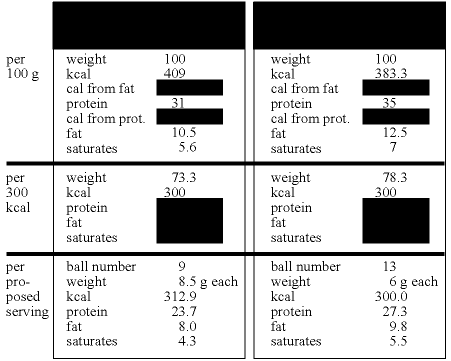

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A method of providing nutrition to an athlete, the method comprising:
providing a bite-size protein-based product; and
providing personalized guidelines for consuming the bite-size protein-based product, wherein the personalized guidelines provide the recommended amount of the bite-size protein-based product to consume before exercising or during recovery after exercising based on at least one of a characteristic of the athlete and a training regimen of the athlete.

2. The method of claim 1, wherein the characteristic of the athlete is selected from the group consisting of weight, height, age, gender and combinations thereof.

3. The method of claim 1, wherein the training regimen is selected from the group consisting of aerobics, weight-lifting, running, swimming, cycling and combinations thereof.

4. The method of claim 1, wherein the personal guidelines are further based on the duration of the training regiment of the athlete.

5. The method of claim 1, wherein the training regimen is determined based on a sport.

6. The method of claim 1, wherein the training regimen is determined based a personal objective.

7. The method of claim 6, wherein the personal objective is selected from the group consisting of weight-loss, muscle building and combinations thereof.

8. The method of claim 1, wherein the personalized guidelines provide the recommended amount of the bite-size protein-based product to consume during exercising based on at least one of the characteristic of the athlete and the training regimen of the athlete.

9. The method of claim 1, wherein the personalized guidelines provide the recommended amount of the bite-size protein-based product to consume before exercising and during recovery after exercising based on at least one of the characteristic of the athlete and the training regimen of the athlete.

10. The method of claim 1, wherein the bite-size protein-based product has a substantially spherical shape.

11. The method of claim 1, wherein the bite-size protein-based product comprises a coating.

12. The method of claim 11, wherein the coating comprises chocolate.

13. The method of claim 11, wherein the coating comprises particulates.

14. The method of claim 1, wherein the bite-size protein-based product is in the shape of a sphere having a diameter ranging from about 12 mm to about 35 mm.

15. The method of claim 1, wherein the bite-size protein-based product weighs from about 5 grams to about 25 grams.

16. The method of claim 1, wherein the bite-size protein-based product comprises a protein content of about 2 grams to about 4 grams.

17. The method of claim 1, wherein the bite-size protein-based product comprises a protein blend comprising soy protein isolates, whey protein isolates and calcium caseinate.

18. The method of claim 1, wherein the bite-size protein-based product comprises an amount of protein ranging from about 20% to about 40% by weight of the bite-size protein-based product.

19. The method of claim 1, wherein the bite-size protein-based product comprises an amount of fat ranging from about 10% to about 40% by weight of the bite-size protein-based product.

20. The method of claim 1, wherein the bite-size protein-based product comprises an amount of protein above 15% by weight together with less than 10% saturated fat by weight of the protein-based product.

21. A method of selling a bite-size protein-based product, the method comprising:
providing a plurality of bite-size protein-based products in a package;
providing personalized guidelines for consuming the bite-size protein-based products, wherein the personalized guidelines provide the recommended amount of the bite-size protein-based product to consume before exercising and during recovery after exercising; and
selling the bite-size protein-based product package and the guidelines to an athlete.

22. The method of claim 21, wherein the personalized guidelines are based on at least one of a characteristic of the athlete and a training regimen of the athlete.

23. A nutritional kit comprising a plurality of bite-size protein-based product and personalized guidelines on how many of the bite-size protein-based products an athlete should consume before exercising and during recovery after exercising.

24. The nutritional kit of claim 23, wherein a plurality of the bite-size protein-based products and the personalized guidelines are together in a package.

25. The nutritional kit of claim 23, wherein the personalized guidelines provide the recommended amount of the bite-size protein-based product to consume before exercising and during recovery after exercising based on a physical characteristic of the athlete.

26. The nutritional kit of claim 23, wherein the personalized guidelines provide the recommended amount of the bite-size protein-based product to the athlete before exercising and during recovery after exercising based on the training regimen of the athlete.

27. A method of enhancing the performance of an athlete, the method comprising:
providing a bite-size protein-based product; and
providing personalized guidelines for consuming the bite-size protein-based product, wherein the personalized guidelines provide the recommended amount of the bite-size protein-based product to consume before the performance based on at least one of a characteristic of the athlete and a training regimen of the athlete.

28. The method of claim 27, wherein the characteristic of the athlete is selected from the group consisting of weight, height, age, gender and combinations thereof.

29. A method of improving recovery of an athlete after exercising, the method comprising:
providing a bite-size protein-based product; and
providing personalized guidelines for consuming the bite-size protein-based product, wherein the personalized guidelines provide the recommended amount of the bite-size protein-based product to consume during recovery after exercising based on at least one of a characteristic of the athlete and a training regimen of the athlete.

30. The method of claim 29, wherein the characteristic of the athlete is selected from the group consisting of weight, height, age, gender and combinations thereof.
